(19) United States
(12) Patent Application Publication
(10) Pub. No.: US 2012/0189960 A1
(43) Pub. Date: Jul. 26, 2012

(54) Hydrophilic Monomer, Hydrophilic Photoresist Composition Containing the same, and Resist Pattern Formation Method

(75) Inventors: Feng-Chin Tang, Yangmei City (TW); Wen-Jen Hsieh, Yangmei City (TW); Chien-Ting Chen, Shulin City (TW)

(73) Assignee: CHUNGHWA PICTURE TUBES, LTD., Yangmei City (TW)

(21) Appl. No.: 13/166,811

(22) Filed: Jun. 23, 2011

(30) Foreign Application Priority Data
Jan. 25, 2011 (TW) 100102734

(51) Int. Cl. G03F 7/20; G03F 7/004; C07C 69/52

(52) U.S. Cl. 430/281.1; 560/196; 560/198; 430/325

(57) ABSTRACT

The present invention is to provide a hydrophilic monomer, and hydrophilic photoresist composition containing the same. The photoresist composition further comprises a hydrophilic resin. The hydrophilic monomer and the hydrophilic resin respectively have a hydrophilic group which is used to react to H2O for the purpose of solving them in pure water. The present invention is also to provide a resist pattern formation method comprising spin coating a hydrophilic photoresist composition on a surface of a substrate to limit a photoresist layer. As a result, the photoresist layer can be developed by pure water. The present invention seeks to overcome the deficiencies in prior art which result in pollution of the environment and cost of photolithography by using basic developing solvent.

### FIELD

The present invention relates to photoresist monomers and photoresist composition containing same. More specifically, it relates to hydrophilic monomers suitable for photolithography processes, hydrophilic photoresist composition containing the same, and resist pattern formation method. The hydrophilic photoresist composition can be developed by pure water, and the composition is suitable for pattern transferring, flat panel display manufacturing and photolithography processes.

### BACKGROUND

In the development of the microelectronic technology, lithography is the most important role in manufacturing processing of semiconductor and TFT-LCD. Most structures of electronic components, for example the pattern and dopant area of thin films, are defined by means of lithography techniques. Briefly speaking, the purpose of lithography techniques (photolithography techniques) is to completely and precisely transfer a geometric pattern to a substrate. First step of lithography process is to coating a photo-sensitive material on a substrate and designing a photomask according to desired pattern. Then, the sequential step, is called exposure, is to emitting parallel light through the photomask and transferring the pattern on the substrate by principle of optical imaging. The light emitted from the source is only able to pass through the transparent area of the photomask and selectively activates the photosensitive material on the surface of the substrate. The pattern formed on the photomask is transferred onto the substrate by development. The photosensitive material coated on the surface of the substrate is called photoresist.

The photoresist composition is the most essential materials to the photolithography process. By coating and exposure of photoresist composition, the desired pattern can be transferred onto the substrate in manufacturing processing of semiconductor or LCD. In general, photoresist is composed of resin, photo-active compound, surfactants (SFA), wherein the resin is used as a Binder and the solvent is used to uniformly mix the substances in photoresist. Over 97 percent of the solvent is vaporized by hard-bake. The surfactants (SFA) are used to improve the adhesive quality of photoresist and the substrate, and the uniformity of coating. Conventionally, after the photo-active compound absorbing the energy of the emitted light in exposure process, it would produce acidic chemicals which can react with alkaline developer in a neutralization reaction.

The purpose of development process is to wash out the exposed photoresist on the surface of the substrate by chemical reaction. In exposure process, the photoresist undergoes a crosslinking reaction or a dissociation reaction. The dissolution rate of the exposed part of the photoresist in developer is much different from the dissolution rate of the unexposed part of the photoresist, and the developer washes away the easier dissolved photoresist for the purpose of development process. In prior art, the developer is typically basic aqueous solutions which may compose of potassium hydroxide (KOH), sodium hydroxide (NaOH) or Sodium carbonate/Sodium Bicarbonate (Na₂CO₃/NaHCO₃) with adequate surfactant. For instance, Taiwan patent number 1324708 disclosed a positive photoresist composition which comprises a substrate composition reacted with acid to increase basic dissolution rate, and an acid generator producing an acid in exposure process. The substrate composition is a Polyphenol compound (a) which comprises more than 2 phenol hydroxyl groups, and its molecular weight is approximately 300-2500 kDa. The Polyphenol compound (a) is selected from the group consisting of I, II, III shown below.

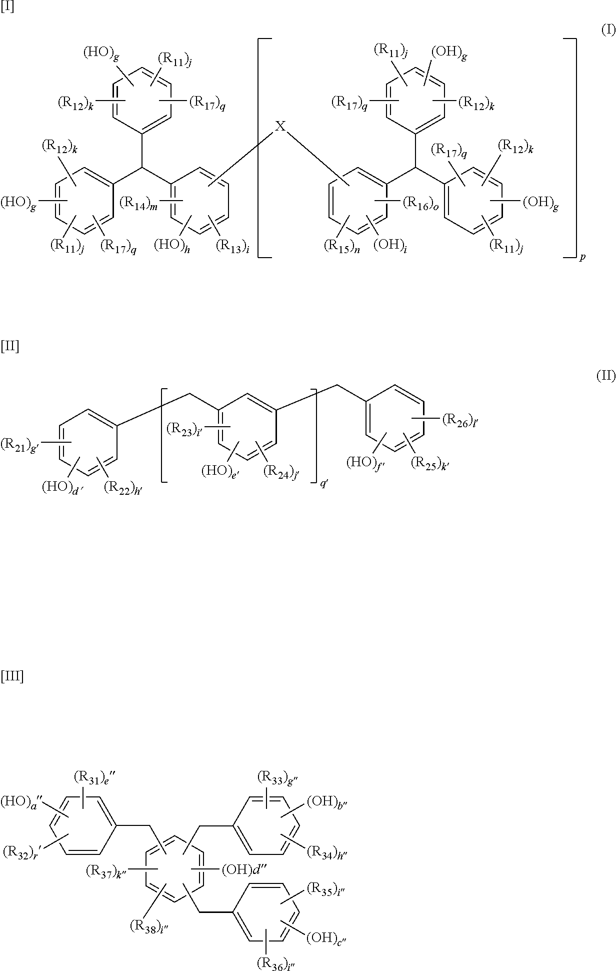

In exposure process, the positive photoresist composition comprising the acid generator undergoes an acidic reaction to dissociate the acid dissociable dissolution inhibiting group, and the substrate composition becomes base-dissolvable.

However, in development process, it is necessary for traditional photoresist composition to react with basic aqueous solutions. The alkaline developer is an environmental hazard, and using the alkaline developer also increases the cost of photolithography process. As a result, a hydrophilic monomer, a hydrophilic photoresist composition containing the same and resist pattern formation method is necessary to overcome deficiencies in prior art, such as high cost of process and using environmental hazardous alkaline developer.

### SUMMARY

In these regards, the present invention is direct toward a hydrophilic monomer suitable for a photoresist. The hydrophilic monomer of the present invention contains a hydrophilic group and is represented by the formula shown below

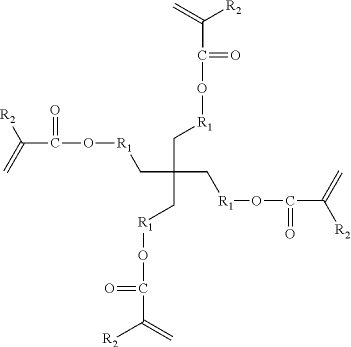

wherein R₁ is —(OC₂H₄)ₙ or —(OC₂H₂NHC₂H₂)ₘ; n and m are each independently an integer from 1 to 20; and R₂ is hydrogen or methyl group.

One purpose of the present invention is to provide a hydrophilic resin suitable for a photoresist. The hydrophilic resin of the present invention contains a hydrophilic group and is represented by the formula shown below

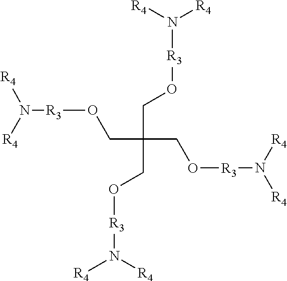

wherein R₃ is selected from the group consisting of —CO(CH₂)ₚ—, —CO(C₂H₄O)_{q}CH₂—, or —CₓH₂ₓ—; p and q are each independently an integer from 1 to 20; x is an integer from 1 to 5; R₄ is selected from the group consisting of —C_{y}H_{2y}COOH, —C_{z}H_{2z}OH, CH₂═CHOOCH₂CH₂—; y is an integer from 1 to 20; and z is an integer from 1 to 6.

Another purpose of the present invention is to provide a hydrophilic photoresist composition comprising a hydrophilic monomer and a hydrophilic resin. Because of the hydrophilic monomer and the hydrophilic resin, the dissolution rate of the exposed part of the photoresist in pure water is much different from the dissolution rate of the unexposed part of the photoresist in exposure process. Pure water is able to washes away the easier dissolved photoresist for the purpose of development process. It also overcomes deficiencies inherent in prior art, such as high cost of process and using environmental hazardous alkaline developer

The other purpose of the present invention is to provide a resist pattern formation method for a hydrophilic photoresist which comprises a step of developing an exposure photoresist layer by pure water. By the resist pattern formation method of the present invention, the deficiency of environmental hazard by using alkaline developer is overcome, the high cost of using alkaline developer is unnecessary, and the efficiency of lithography process is increased.

### BRIEF DESCRIPTION OF THE DRAWINGS

FIG. 1 is shows a photoresist pattern obtained by the resist pattern method of an embodiment of the present invention and observed by optical microscope.

### DETAILED DESCRIPTION

The present invention discloses a hydrophilic monomer represented by the following formula (I)

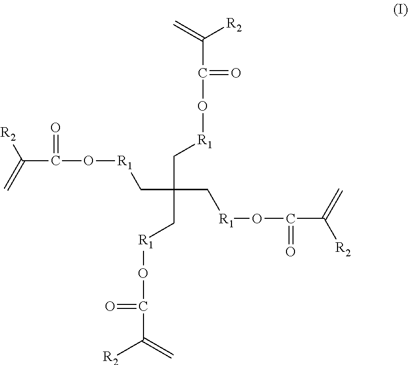

wherein R₁ is —(OC₂H₄)ₙ or —(OC₂H₂NHC₂H₂)ₘ; n and m are each independently an integer from 1 to 20; and R₂ is hydrogen or methyl group. In one embodiment of the present invention, n is an integer from 1 to 15 and m is an integer from 1 to 17. In one preferred embodiment, n is an integer from 1 to 10 and m is an integer from 3 to 15.

The hydrophilic monomer of the present invention is suitable for a photoresist and contains a hydrophilic group which is able to react with pure water (H₂O) to dissolve the hydrophilic monomer in pure water.

The present invention discloses a hydrophilic resin represented by the following formula (II)

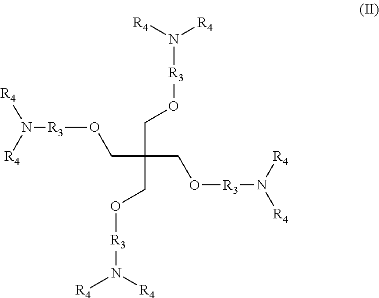

wherein R₃ is selected from the group consisting of —CO(CH₂)ₚ—, —CO(C₂H₄O)_{q}CH₂—, or —CₓH₂ₓ—; p and q are each independently an integer from 1 to 20; x is an integer from 1 to 5; R₄ is selected from the group consisting of —C_{y}H_{2y}COOH, —C_{z}H_{2z}OH, CH₂═CHCOOCH₂CH₂—; y is an integer from 1 to 20; and z is an integer from 1 to 6. In one embodiment of the present invention, p is an integer from 1 to 15, q is an integer from 2 to 17, x is an integer from 1 to 5, y is an integer from 1 to 15, and z is an integer from 1 to 6. In one preferred embodiment, p is an integer from 1 to 10, q is an integer from 2 to 15, x is an integer from 1 to 5, y is an integer from 1 to 10, and z is an integer from 1 to 6. The hydrophilic resin of the present invention is useful for a photoresist. The hydrophilic resin contains a hydrophilic group which is able to react with pure water (H₂O) to dissolve the hydrophilic resin in pure water.

The present invention discloses a hydrophilic photoresist composition comprising a hydrophilic monomer. In one embodiment of the present invention, the hydrophilic photoresist composition includes a hydrophilic monomer of formula (I) and a hydrophilic resin of formula (II) described above. The hydrophilic photoresist composition further includes a photo-initiator, an additive and a solvent, wherein the photo-initiator is used to absorb the specific wavelength incident radiation from light source to generate free radicals, and forms a polymer having high molecular weight by promoting polymerization. The above process, so-called “Photopolymerization”, is to promote polymerization by light radiation. The additive is used to adjust the photochemical property of the photoresist composition in exposure process, such as adhesion, stability and contractility, for the purpose of achieving optimum pattern resolution. The solvent is used to mix the substance of the photoresist evenly to reduce viscosity of the photoresis. By the solvent, the thickness and smoothness of photoresist coating can be adjusted, the adhesion between photoresist and substrate can be improved, and the evenness of photoresist coating is increased. In one embodiment, the photo-initiator in the hydrophilic photoresist composition of the present invention comprises, but not limit to, 2-benzyl-2-dimethylamino-1-(4-morpholinphenyl)butanone (so-called BDMB, Irgacure 369, or 1-369); the additive comprises, but not limit to, megafatuk R-08; and the solvent comprises, but not limit to, the mixture of propylene glycol monomethyl ether (PGME) and propylene glycol monomethylether acetate (PGMEA). The photopolymerization promoted by the hydrophilic photoresist composition of the present invention is schematically represented below:

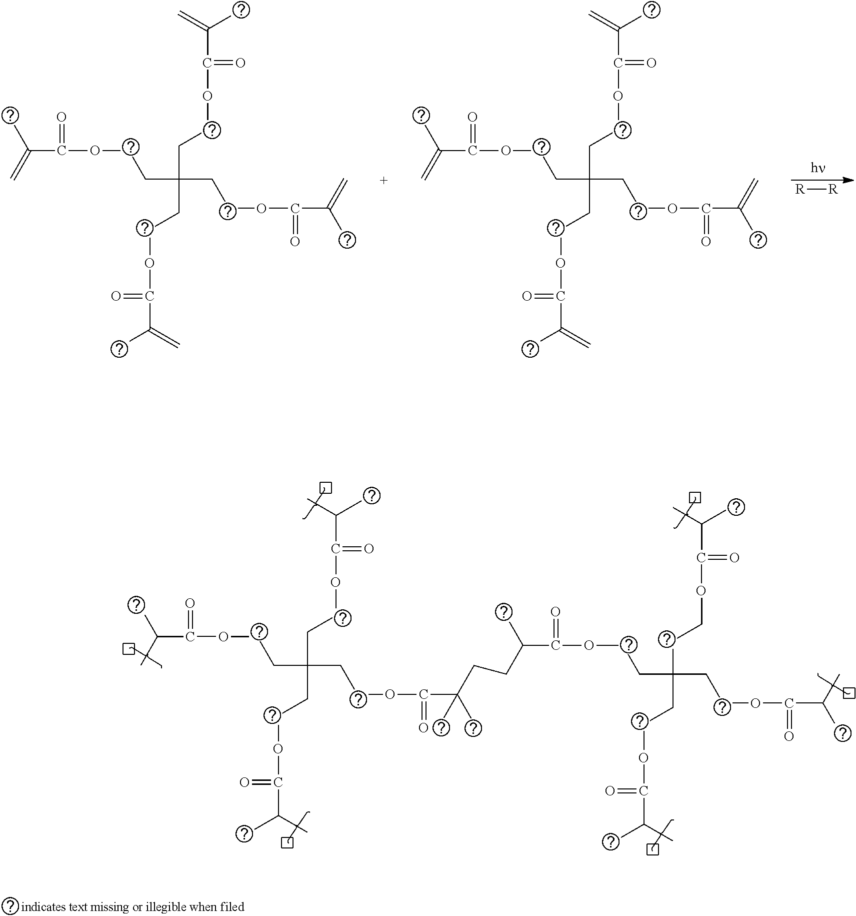

wherein R—R represents the photo-initiator comprising, but not limit to, 2-benzyl-2-dimethylamino-1-(4-morpholinphenyl)butanone (so-called BDMB; Irgacure 369, and 1-369).

At this time, the unphotopolymerized hydrophilic monomer and resin is dissolved and removed by pure water in development process because of the hydrophilic group contained in the monomer and resin.

In the preparation of the hydrophilic photoresist composition of the present invention, the percentage of the components of the composition is based on the percent by total weight of the photoresist composition. The photoresist composition is prepared by mixing the components which have been weighted. In one embodiment, the concentration of the hydrophilic monomer of formula (I) in the hydrophilic photoresist composition is about 10 to 20 weight percent, preferably 16 weight percent; and the concentration of the hydrophilic resin of formula (II) is about 15 to 25 weight percent, preferably 17 weight percent. The concentration of the photo-initiator is about 0.5 to 3 weight percent; the concentration of the additive is about 2 to 3 weight percent; and the concentration of the solvent is about 55 to 75 weight percent. In one preferred embodiment, the photo-initiator preferably comprises 2-benzyl-2-dimethylamino-1-(4-morpholinphenyl)butanone (so-called BDMB, Irgacure 369, or 1-369) and is 2.2 weight percent; the additive preferably comprises megafatuk R-08 (product by Dainippon Ink and Chemicals, Inc.) which is a fluoroacrylate ester copolymer and is preferably 2 weight percent; and the solvent preferably comprises the mixture of propylene glycol monomethyl ether (PGME) and propylene glycol monomethylether acetate (PGMEA) and is 62.8 weight percent. The hydrophilic photoresist composition of the present invention is applied by various methods known in the art. The method of coating the hydrophilic photoresist composition on a substrate includes spin coating, dip coating, roll coating or any other coating methods known in the art. When using spin coating method, the concentration of solid substance in the photoresist composition can be adequately adjusted to form a desired thickness of photoresist layer according to the spin-coating apparatus used, viscosity of the photoresist composition, the rotation speed of the spin-coating apparatus, and spin time.

The present invention discloses a resist pattern formation method comprising steps of: spin-coating a hydrophilic photoresist composition on a surface of a substrate to form a photoresist layer; drying the photoresist layer; first baking the photoresist layer; exposing the photoresist layer through a photomask to incident radiation; developing the photoresist layer by pure water; and second baking the photoresist layer. The hydrophilic photoresist composition of the present invention is suitable for coating on various substrates known in the art, such as the panel substrate for display, microprocessor, and silicon wafer or silicon wafer with a silicon dioxide (SiO2) layer used in integrated circuit elements. It is also suitable for applied on the substrate made of aluminum/aluminum oxide, gallium arsenide (GaAs), ceramics, quartz, copper or glass. In one embodiment, in the resist pattern formation method according to the present invention, the step of spin-coating the hydrophilic photoresist composition is at 400 to 1000 rpm; and vacuum drying at about 2 ton pressure. The resist pattern formation method of the present invention provides an embodiment wherein the first baking occurs at a temperature of 90° C. to 120° C.; and the second baking occurs at 230° C. for 30 minutes. In another embodiment, the resist pattern formation method comprises the steps wherein exposing the photoresist layer at 50 mJ to 200 mJ incident radiation; and developing the photoresist layer for 15 to 45 seconds.

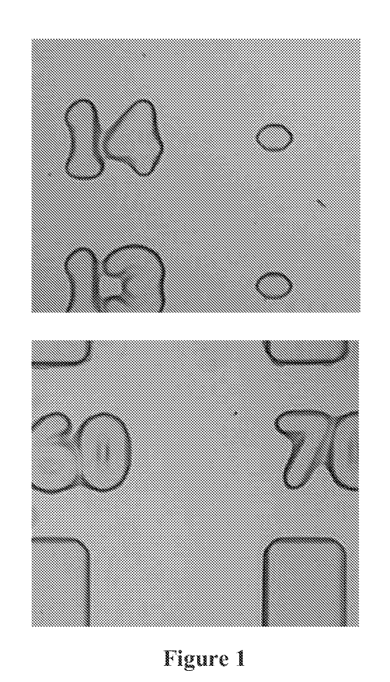

Please refer to FIG. 1, it shows a photoresist pattern obtained by the above method of the present invention and observed by optical microscope. In contrast with the traditional method, the resist pattern formation method disclosed in the present invention provides photoresist patterns with a higher resolution, and also provides a step of developing the photoresist layer by pure water. The present invention overcomes the drawbacks of environmental hazard by using alkaline developer and high cost of using alkaline developer. As a result, the efficiency of lithography process can be increased.

In the above description, for the purposes of explanation, numerous specific details are set forth in order to provide a thorough understanding of the structure and process of the present invention. Those skilled in the art readily will appreciate that the scope of the present invention is not limited to the described preferred embodiments. The scope of the present invention is expressly not limited but expected as specified in the accompanying claims. Various changes and modifications can be made within the spirit and scope of the present invention, as defined by the following Claims. Therefore, the present invention can be widely applied to other embodiments.

What is claimed is:

1. A hydrophilic monomer represented by the following formula (I) 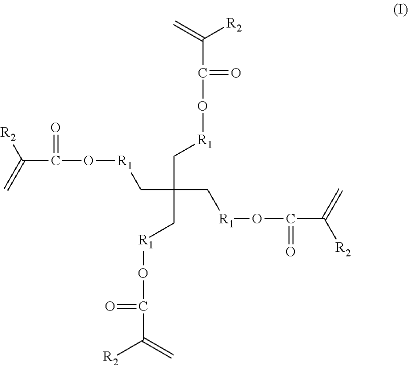
wherein
R₁ is —(OC₂H₄)ₙ or —(OC₂H₂NHC₂H₂)ₘ;
R₂ is hydrogen or methyl group; and
n and m are each independently an integer from 1 to 20.

2. The hydrophilic monomer according to claim 1, wherein n is an integer from 1 to 15 and m is an integer from 1 to 17.

3. A hydrophilic photoresist composition comprising the hydrophilic monomer according to claim 1.

4. The hydrophilic photoresist composition according to claim 3 further comprising a hydrophilic resin represented by the following formula (II) 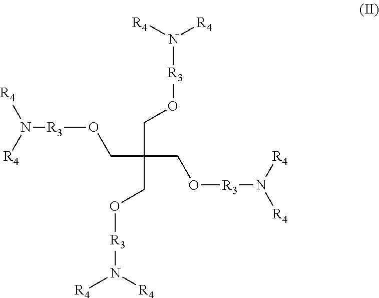
wherein
R₃ is selected from the group consisting of —CO(CH₂)ₚ—, —CO(C₂H₄O)_{q}CH₂—, or —CₓH₂ₓ—;
R₄ is selected from the group consisting of —C_{y}H_{2y}COOH, —C_{z}H_{2z}OH, CH₂═CHCOOCH₂CH₂—;
p and q are each independently an integer from 1 to 20;
x is an integer from 1 to 5;
y is an integer from 1 to 20; and
z is an integer from 1 to 6.

5. The hydrophilic photoresist composition according to claim 4, wherein p is an integer from 1 to 15, q is an integer from 2 to 17, x is an integer from 1 to 5, y is an integer from 1 to 15, and z is an integer from 1 to 6.

6. The hydrophilic photoresist composition according to claim 3 further comprising a photo-initiator, an additive and a solvent, wherein the additive is used to adjust the photochemical property of the hydrophilic photoresist composition in exposure process.

7. The hydrophilic photoresist composition according to claim 6, wherein the hydrophilic resin is 15 to 25 weight percent, and the hydrophilic monomer is 10 to 20 weight percent.

8. The hydrophilic photoresist composition according to claim 7, wherein the photo-initiator is 0.5 to 3 weight percent; the additive is 2 to 3 weight percent; and the solvent is 55 to 75 weight percent.

9. The hydrophilic photoresist composition according to claim 7, wherein the photo-initiator comprises 2-benzyl-2dimethylamino-1-(4-morpholinphenyl)butanone; the additive comprises megafatuk R-08; and the solvent comprises the mixture of propylene glycol monomethyl ether (PGME) and propylene glycol monomethylether acetate (PGMEA).

10. The hydrophilic photoresist composition according to claim 10, wherein hydrophilic resin is 17 weight percent; the hydrophilic monomer is 16 weight percent; the photo-initiator is 2.2 weight percent; the additive is 2 weight percent; and the solvent is 62.8 weight percent.

11. A resist pattern formation method comprising the steps of:
coating a hydrophilic photoresist compositions according to claims 3 to 10 on a surface of a substrate to form a photoresist layer;
first baking the photoresist layer;
exposing the photoresist layer to incident radiation;
developing the photoresist layer by pure water; and
second baking the photoresist layer.

12. The resist pattern formation method according to claim 11, wherein said step of coating the hydrophilic photoresist compositions on the surface of the substrate is spin-coating.

13. The resist pattern formation method according to claim 12, wherein said step of spin-coating the hydrophilic photoresist composition is at 400 to 1000 rpm.

14. The resist pattern formation method according to claim 12, wherein said step of first baking occurs at a temperature of 90° C. to 120° C.; and said step of second baking occurs at 230° C. for 30 minutes.

15. The resist pattern formation method according to claim 12, wherein said step of exposing the photoresist layer is at 50 mJ to 200 mJ incident radiation.

16. The resist pattern formation method according to claim 12, further comprising a step of vacuum drying before said step of first baking the photoresist layer.

17. The resist pattern formation method according to claim 16, wherein said step of vacuum drying is promoted at 2 torr pressure.
